(19) United States
(12) Patent Application Publication
(10) Pub. No.: US 2018/0033985 A1
(43) Pub. Date: Feb. 1, 2018

(54) An Electron Transport Layer Material and the Application Thereof

(71) Applicant: South China University of Technology, Guangzhou City (CN)

(72) Inventors: Fei HUANG, Guangzhou (CN); Chen SUN, Guangzhou City (CN); Zhihong WU, Guangzhou City (CN); Hin Lap YIP, Guangzhou City (CN); Yong CAO, Guangzhou City (CN)

(73) Assignee: South China University of Technology, Guangzhou City (CN)

(21) Appl. No.: 15/550,514

(22) PCT Filed: Mar. 4, 2016
(86) PCT No.: PCT/CN2016/075708

§ 371 Date: Aug. 11, 2017

(30) Foreign Application Priority Data
Mar. 6, 2015 (CN) 201510100372.6
Mar. 30, 2015 (CN) 201510145506.6

(51) Int. Cl. H01L 51/44; H01L 51/00; H01L 51/50; C07D 401/04

(52) U.S. Cl. CPC H01L 51/44; C07D 401/04; H01L 51/0035; H01L 51/0032; H01L 51/50

(57) ABSTRACT

The present invention belongs to the technical field of optoelectronic devices, and discloses an electron transport layer material and its application. This material has the following structure:

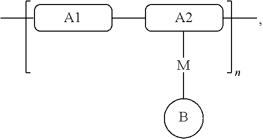

wherein n is a natural number of 1 to 10000, B is a strongly polar group, A1 and A2 are the same or different aromatic ring derivatives or conjugated units containing carbon-carbon double bonds and carbon-nitrogen bonds, M is a connection unit between A2 and B and is an alkyl group containing 1 to 20 carbon atoms, or is an alkyl group in which one or more carbon atoms are replaced by one or more functional groups selected from oxygen atoms, alkenyl groups, alkynyl groups, aryl groups or ester groups, and the hydrogen atom is replaced by a fluorine atom, a chlorine atom, a bromine atom, an iodine atom or the above-mentioned functional groups.

### FIELD OF THE INVENTION

The present invention belongs to the technical field of optoelectronic devices, and especially relates to an electron transport layer material and the application thereof.

### BACKGROUND OF THE INVENTION

In recent years, energy consumption is increasing, while reserves of coal, petroleum, natural gas and other traditional energy are limited, thus people pay more and more attention to the development of clean energy with solar energy as the representative. Organic optoelectronic materials and devices are very suitable for industrial production and have a very broad commercial prospect, due to their advantages of easily available materials, low-temperature solvent processing, good mechanical properties and large-area manufacturing. Since C. W. Tang's research group in Kodak Company of the US proposed organic small-molecule thin-film electroluminescent devices in 1987 [Tang, C. W.; Van Slyke S. A. et al.; Applied Physics Letters, 1987, 51, 913.] and R. H. Friend's research group in University of Cambridge of the UK proposed organic polymer thin-film electroluminescent devices in 1990 [Burroughes J. H., Bradley D. C., Brown A. R., et al. Nature, 1990, 347: 539-541.], the organic flat panel display technology has made great progress, which has entered the stage of industrialization to become a next-generation product as a replacement for the liquid crystal display. At the same time, organic solar cells, organic field-effect transistors, organic biology, chemical sensors and other fields of organic optoelectronics have also developed vigorously. To date the efficiency of organic solar cells (OSCs) has surpassed 12% (http://www.orgworld.de) through the continuous efforts of scientific research workers, and therefore the OSCs are highly valued by the industry and have a bright marketization prospect.

At the same time, the solution-processing organic-inorganic hybrid perovskite solar cells have recently received much attention around the world. These perovskite materials possess unique prOperties of strong absorption, high mobility, long carrier lifetime, controllable bandgap and various of processing methods. In 2009, Tsutomu Miyasaka's research group first prepared a dye-sensitized solar cell using a perovskite-based organic-inorganic hybrid material as the photoactive layer, yielding efficiency of 3.81% (J. Am. Chem. Soc. 2009, 131, 6050). In just a few years, the power conversion efficiency of the small-area laboratory device has been increased from 3.81% to over 20%, making it a good candidate for solar cell technology.

In order to obtain efficient OSC devices, it is important to efficiently extract electrons and holes to the cathode and anode, respectively. Therefore, many efficient OSC devices are of a multi-layer device structure, that is, in addition to the intermediate active layer, one or more layers of the hole transport layer or electron transport layer are required. Consequently, in addition to developing excellent active layer materials, the development of the excellent electron transport layer and hole transport layer is also the key to achieve efficient organic thin-film battery devices.

Previous studies have found that conjugated polyelectrolytes and their neutral precursors are very good electron transport layers [Chem. Mater. 2004, 16, 708; J. Am. Chem. Soc. 2004, 126, 9845-9853; Chinese Patent No. ZL200310117518.5], wherein such conjugated polyelectrolyte materials can be used not only in light emitting devices, but also as an interface modification layer to greatly improve the performance of OSCs, field effect transistors and perovskite solar cells [Chem. Soc. Rev. 2010, 39, 2500]. However, the lowest unoccupied molecular orbital (LUMO) energy level of these conjugated polyelectrolyte material is too high, and the electron mobility is low, so that the optimal thickness of these materials in optoelectronic devices is only 5 nm, which is more demanding for the device processing technology.

### SUMMARY OF THE INVENTION

In order to overcome the above-mentioned shortcomings and deficiencies of the prior art, a primary purpose of the present invention is to provide a kind of electron transport layer materials, which can greatly improve the open circuit voltage and device performance of the solar cells.

A further purpose of the present invention is to provide the application of the above-mentioned electron transport layer materials in optoelectronic devices.

The purposes of the present invention are achieved through the following technical solution:

An electron transport layer material is provided, having the following structure:

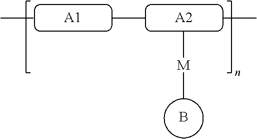

Wherein n is a natural number of 1 to 10,000. B is a strongly polar group. A1 and A2 are the same or different aromatic ring derivatives or conjugated units containing carbon-carbon double bonds and carbon-nitrogen bonds. M is a connection unit between A2 and B, and it is an alkyl group containing 1 to 20 carbon atoms, or is an alkyl group in which one or more carbon atoms are replaced by one or more functional groups selected from oxygen atoms, alkenyl groups, alkynyl groups, aryl groups or ester groups, and the hydrogen atom is replaced by a fluorine atom, a chlorine atom, a bromine atom, an iodine atom or the above-mentioned functional groups.

The strongly polar group is selected from one or more of the groups consisting of an amine group, a quaternary ammonium salt group, a phosphate radical, a phosphate group, a sulfonate radical, a carboxyl group and a hydroxyl group.

A1 and A2 are of one or more of the following structures:

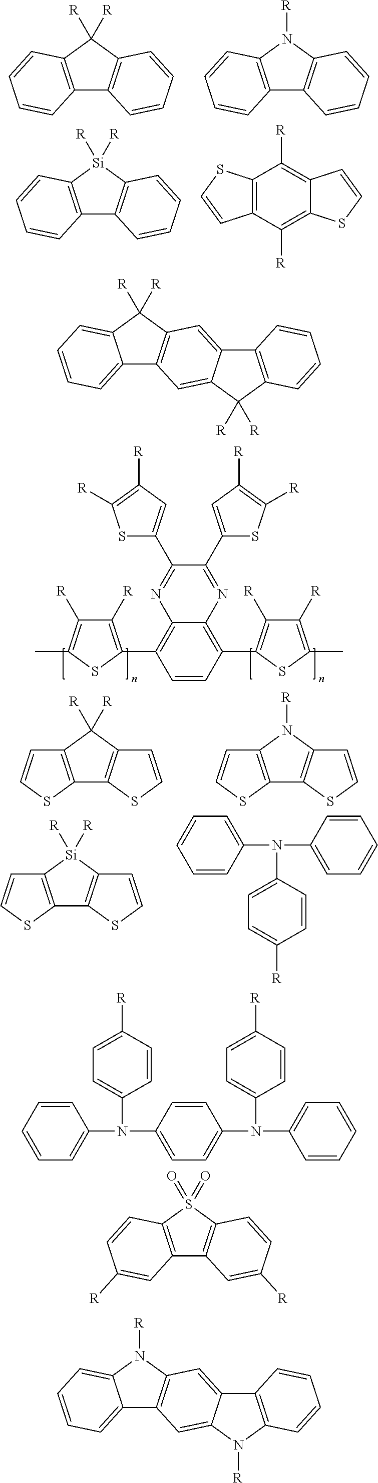

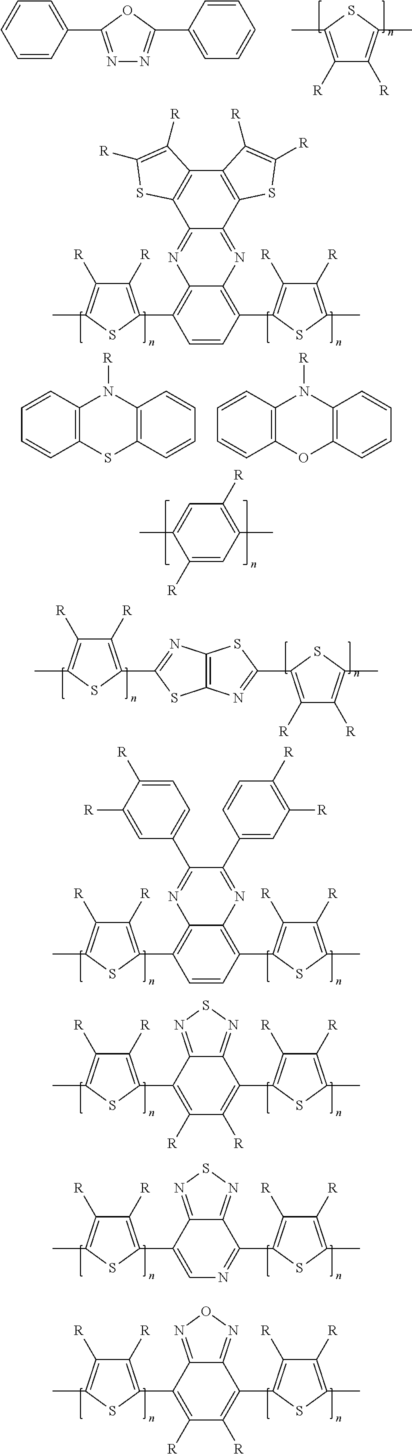

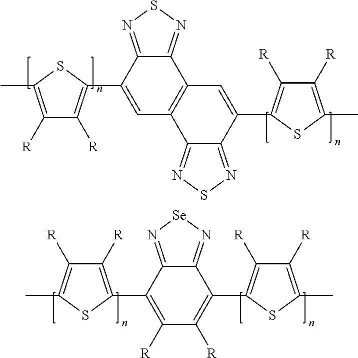

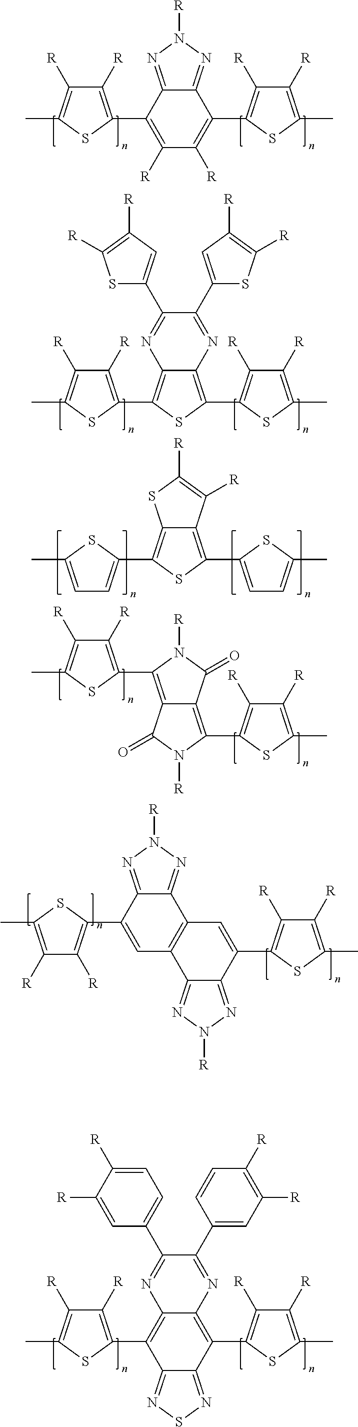

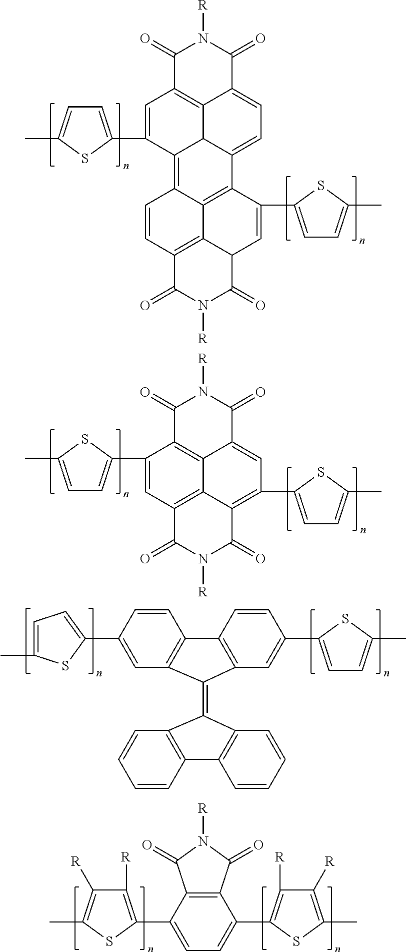

Wherein n is a natural number of 1 to 10,000. R is an alkyl group containing 1 to 20 carbon atoms, or it is an alkyl group in which one or more carbon atoms are replaced by one or more functional groups selected from oxygen atoms, alkenyl groups, alkynyl groups, aryl groups or ester groups, and the hydrogen atom is replaced by a fluorine atom, a chlorine atom, a bromine atom, an iodine atom or the above-mentioned functional groups.

Preferably, A1 is

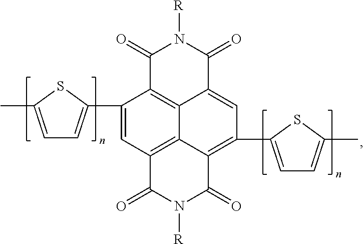

wherein n is a natural number of 1 to 3, R is an alkyl group containing 1 to 20 carbon atoms, or is an alkyl group in which one or more carbon atoms are replaced by one or more functional groups selected from oxygen atoms, alkenyl groups, alkynyl groups, aryl groups or ester groups, and the hydrogen atom is replaced by a fluorine atom, a chlorine atom, a bromine atom, an iodine atom or the above-mentioned functional groups; A2 is of one or more of the following structures:

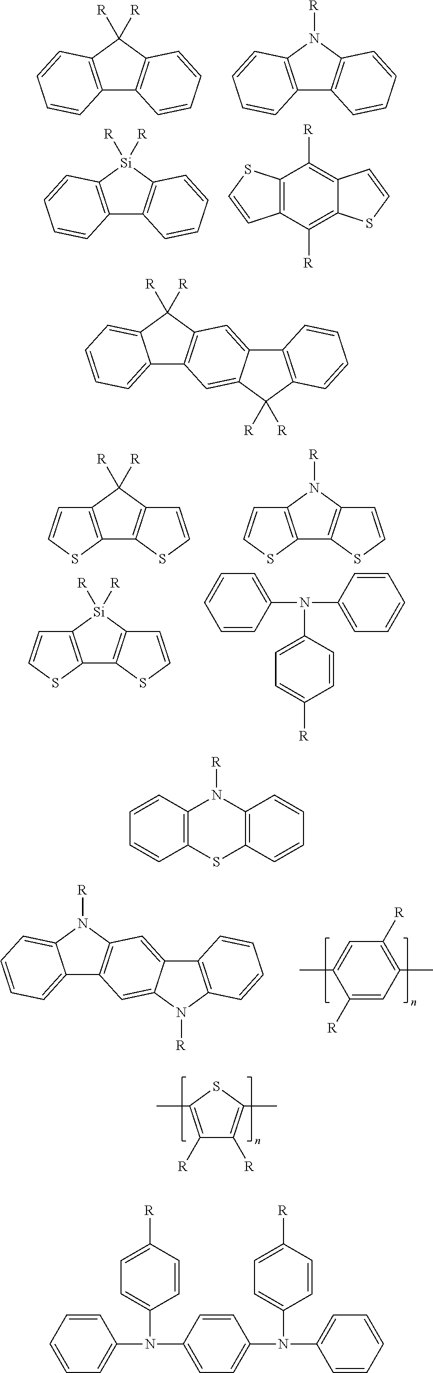

Wherein n is a natural number of 1 to 10000, R is an alkyl group containing 1 to 20 carbon atoms, or is an alkyl group in which one or more carbon atoms are replaced by one or more functional groups selected from oxygen atoms, alkenyl groups, alkynyl groups, aryl groups or ester groups, and the hydrogen atom is replaced by a fluorine atom, a chlorine atom, a bromine atom, an iodine atom or the above-mentioned functional groups.

When A1 and A2 are of the above-mentioned preferred structures, the material of the electron transport layer can be prepared by the following method: First mixing the A2 monomers containing a polar group and the monomers of the naphthalimide ring in an equimolar amount to obtain a polymer in which the polar groups are not ionized under the action of an organic base and a palladium catalyst, then subjecting the resulting polymer to a salinization reaction at room temperature in the dark to obtain a polymer in which the polar groups are ionized, and then further treating the resulting ionized polymer with an ion exchange resin containing different pairs of ions and finally subjecting it to separation to obtain the electron transport layer material.

The above-mentioned electron transport layer material can be applied to optoelectronic devices.

Compared with the prior art, the present invention has the following advantages and beneficial effects:

(1) In the present invention, the conjugated polymer containing a strongly polar group is used in the electron transport layer, wherein the polymer containing a strongly polar group side chain can significantly modify the cathode interface and improve the carrier transport capability, and thus good device performance is achieved.

(2) The conjugated polymer material containing an electron-withdrawing group such as a naphthalimide ring has a LUMO energy level matched with the electron acceptor material in the active layer, and is thus more advantageous for the electrons to be transferred from the acceptor material in the active layer to the electron transport layer. It can be applied to an optoelectronic device such as a light-emitting device and a photovoltaic device as an electron transport layer with good charge transport ability, improving the processing technology and performance of the device.

### BRIEF DESCRIPTION OF THE DRAWINGS

FIGS. 1A & B is a Fourier infrared absorption diagram of the polymers obtained in Examples 1 to 4, wherein FIG. 1A shows Fourier infrared absorption spectra of the polymers PNDIT-F6N, PNDIT-F6N-I and PNDIT-F6N-OH; and FIG. 1B shows Fourier infrared absorption spectra of the polymers PNDIT-F3N, PNDIT-F3N-I and PNDIT-F3N-OH.

FIG. 2 is a graph showing the thermogravimetric analysis of the polymers obtained in Examples 1 to 4.

FIG. 3 shows an absorption spectrum of the polymers obtained in Examples 1 to 4 in a methanol solution.

FIG. 4 shows a thin-film absorption spectrum of the polymers obtained in Examples 1 to 4.

FIG. 5 is a graph comparing the reduction potential curves of the polymers obtained in Examples 1-4.

FIG. 6 is a graph comparing the oxidation potential curves of the polymers obtained in Examples 1-4.

FIG. 7 is J-V curves of organic solar cells prepared by using the polymers obtained in Examples 1 to 4 as the electron transport layer.

FIG. 8 is a characterization diagram of the single-electron device of the polymers (PNDIT-F6N, PNDIT-F3N) obtained in Examples 1 and 2.

FIG. 9 is an ultraviolet-visible absorption spectrum of a polymer solution and a thin film obtained in Example 5.

FIG. 10 is J-V curves of the organic-inorganic planar heterojunction perovskite solar cell (with CH₃NH₃PbI₃₋ₓClₓ as the photoactive layer) using a different electron transport layer in Example 7.

### DETAILED DESCRIPTION OF THE INVENTION

The present invention will be further described in detail below with reference to examples and drawings; however, the embodiments of the present invention are not limited thereto.

The practice of the present invention may employ conventional techniques of polymer chemistry within the skill of the art. In the following examples, efforts should be made to ensure the accuracy of the numbers used (including quantity, temperature, reaction time, etc.); however, some experimental errors and deviations should be considered. The temperatures used in the examples below are in degrees Celsius, and the pressure is at or near atmospheric pressure. All the solvents were purchased at the analytical or chromatographic grade, and all the reactions were carried out in an inert atmosphere of argon. Unless otherwise indicated, all the reagents were obtained commercially.

### Example 1

### Preparation of poly {2,7-[9,9′-bis(N,N-diethylhexyl-6-amino)fluorene]-co-5,5′-[2,6-(bis-2-thienyl)-N,N′-diisooctyl-1,4,5,8-naphthalimide]}(PNDIT-F6N)

The chemical reaction process is shown below, including the following specific reaction steps and reaction conditions:

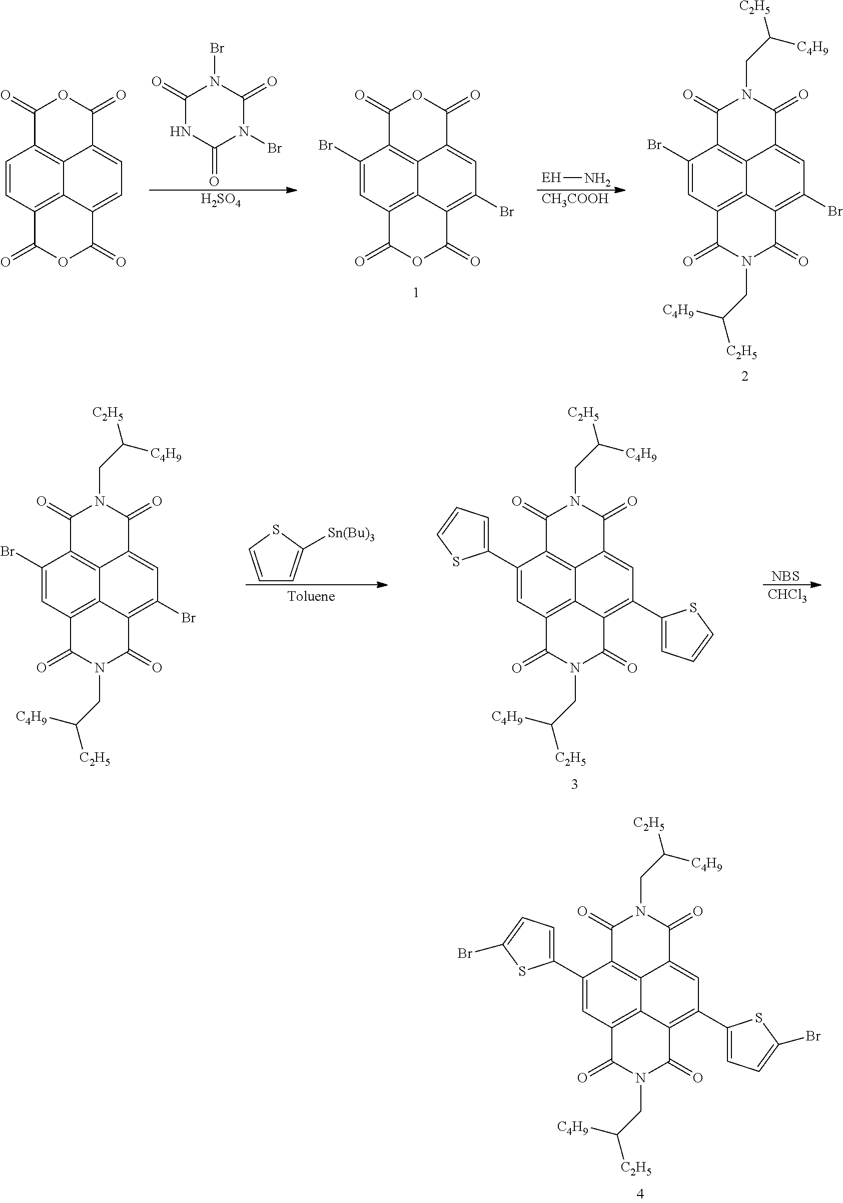

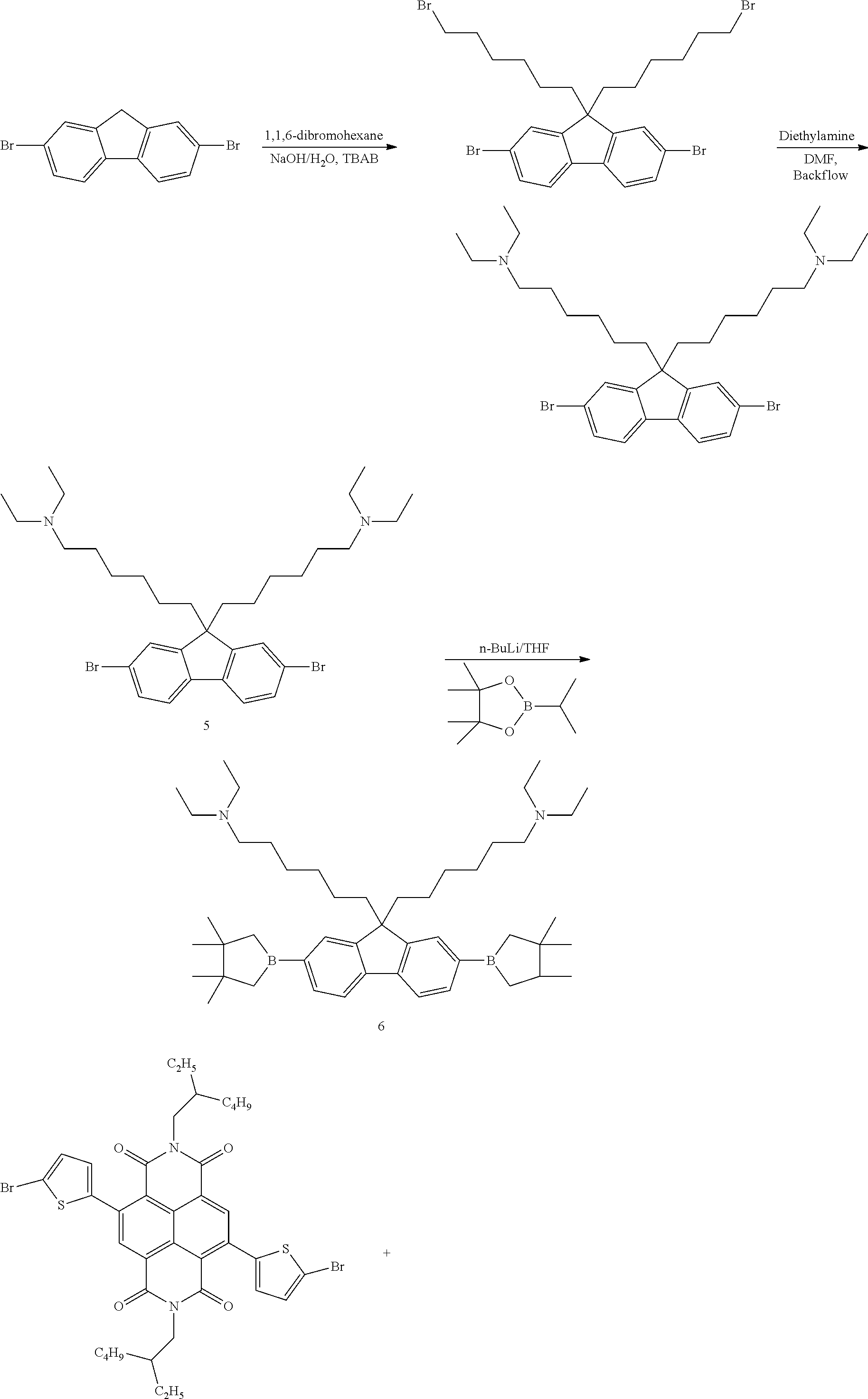

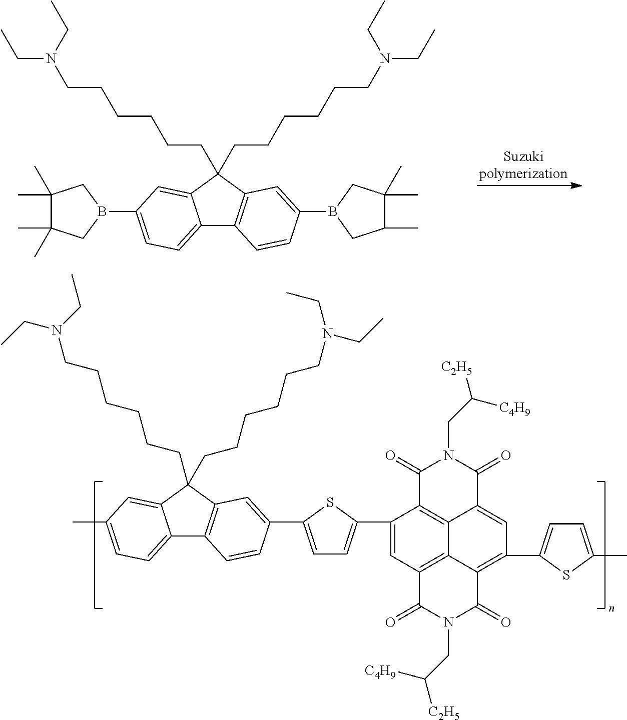

(1) The monomer 2,7-bis(trimethylene borate)-9,9′-bis(N,N-diethylhexyl-6-amino)fluorene was prepared according to the method disclosed in the literature (Adv. Mater., 2011, 23, 1665). The monomer 2,6-(bis-5-bromo-2-thienyl)-N,N′-diisooctyl-1,4,5,8-naphthalimide was prepared according to the method disclosed in the literature (Chem. Mater., 2011, 23, 4563). The specific steps are as follows: Adding 1.944 g (3 mmol) of the monomer 2,6-dibromo-N,N′-diisooctyl-1,4,5,8-naphthalimide (prepared according to the method disclosed in the patent [PCT WO2011/144537 A1]) to a 100 mL two-necked flask equipped with a stir bar and bubbling nitrogen for 10 min, adding 40 mL of clean toluene to the reaction flask and stirring it to get dissolved, adding 2.5 g (6.1 mmol) of thiophene tributyltin under the protection of nitrogen (prepared according to the method disclosed in the literature [Synthetic Metals 2006, 156 (2-4), 166-175]), then adding the catalyst of (beta-4)-platinum, heating to 90° C. with stirring, and reacting for 5 h. After completion of the reaction, pouring the mixture into an aqueous solution of ammonium chloride, extracting with dichloromethane, drying, leaching and concentrating, eluting the resulting solid with dichloromethane petroleum ether (at a volume ratio of 1:1) as the eluent, passing a silica gel column, and recrystallizing the resulting solid with methanol-chloroform to obtain 1.7 g of a red pure product at a yield of 86.7%. Adding 1.308 g (2 mmol) of the product obtained in the above step to a 250 mL two-necked flask equipped with a stir bar, adding 20 mL of DMF and 60 mL of chloroform and stirring them to get dissolved, then dissolving 0.7832 g of NBS in a mixed solvent of 20 mL of chloroform and 40 mL of DMF, slowly adding the solution dropwise to the reaction flask with an ice bath, and reacting in the dark for two days. After completion of the reaction, concentrating the mixture directly into a solid, eluting the resulting solid with dichloromethane petroleum ether (at a volume ratio of 1:1) as the eluent, passing a silica gel column, and recrystallizing the resulting solid with methanol-chloroform to obtain 1.6 g of a red needle crystal, i.e., the monomer 2,6-(bis-5-bromo-2-thienyl)-N,N′-diisooctyl-1,4,5,8-naphthalimide, at a yield of 96%.

(2) Adding 0.406 g of the monomer 2,6-(bis-5-bromo-2-thienyl)-N,N′-diisooctyl-1,4,5,8-naphthalimide and 0.364 g of the monomer 2,7-bis(trimethylene borate)-9,9′-bis(N,N-diethylhexyl-6-amino)fluorene to a 15 mL thick-walled pressure-proof pipe equipped with a stir bar, adding 1 mL of 20% aqueous tetrabutylammonium hydroxide solution, adding 2 mL of purified tetrahydrofuran and 4 mL of clean toluene, adding 25 mg of the catalyst tetrakis(triphenylphosphine)palladium, bubbling nitrogen for 20 min, sealing, heating to 110° C. with stirring and reacting for 1 h, precipitating the reaction solution into methanol to obtain a crude product, filtering, drying, then washing the polymer with acetone in a Soxhlet extractor for 24 h, and then extracting 0.532 g of the target polymer with chloroform at a yield of 89.7%. Mₙ=17000, and PDI=1.6.

### Example 2

### Preparation of poly{2,7-[9,9′-bis(N,N-dimethylpropyl-3-amino)fluorene]-co-5,5′-[2,6-(bis-2-thienyl)-N,N′-diisooctyl-1,4,5,8-naphthalimide]}(PNDIT-F3N)

The chemical reaction process is shown below, including the following specific reaction steps and reaction conditions:

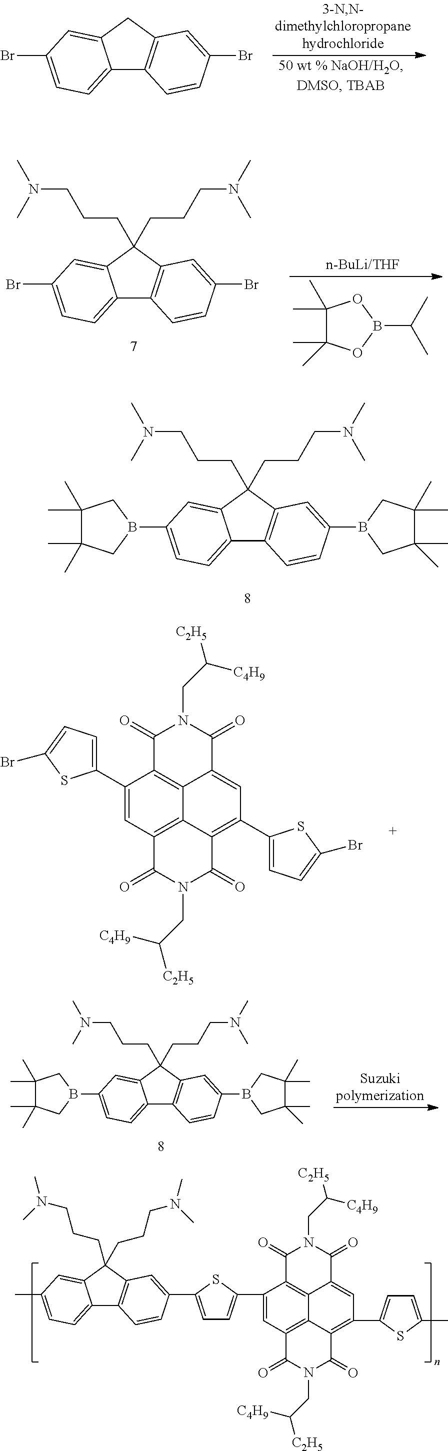

Adding 0.406 g of the monomer 2,6-(bis-5-bromo-2-thienyl)-N,N′-diisooctyl-1,4,5,8-naphthalimide (the preparation method is the same as that of Example 1) and 0.294 g of the monomer 2,7-bis(trimethylene borate)-9,9′-bis(N,N-dimethylpropyl-3-amino)fluorene (prepared according to the method disclosed in the literature [J. Am. Chem. Soc. 2004, 126, 9845-9853]) to a 15 mL thick-walled pressure-proof pipe equipped with a stir bar, adding 1 mL of 20% aqueous tetrabutylammonium hydroxide solution, adding 2 mL of purified tetrahydrofuran and 4 mL of clean toluene, adding 25 mg of the catalyst tetrakis(triphenylphosphine)palladium, bubbling nitrogen for 20 min, sealing, heating to 110° C. with stirring and reacting for 1 h, precipitating the reaction solution into methanol to obtain a crude product, filtering, drying, then washing the polymer with acetone in a Soxhlet extractor for 24 h, and then extracting 0.497 g of the target polymer with chloroform at a yield of 94.3%. Mₙ=25000, and PDI=1.4.

### Example 3

### Preparation of poly {2,7-[9,9′-bis(N,N-diethylhexyl-6-hydroxylamine)fluorene]-co-5,5′-[2,6-(bis-2-thienyl)-N, N′-diisooctyl-1,4,5,8-naphthalimide]} (PNDIT-F6N-OH)

The chemical reaction process is shown below, including the following specific reaction steps and reaction conditions:

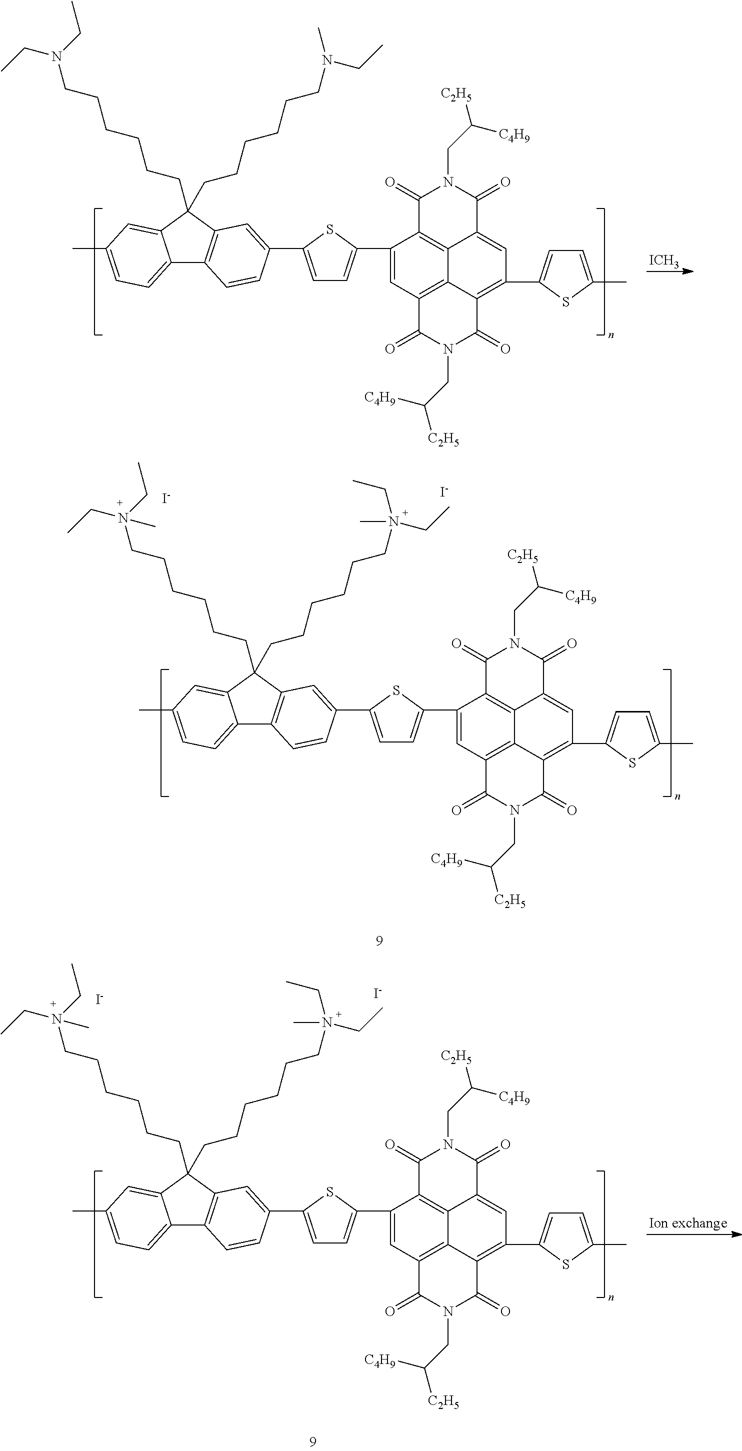

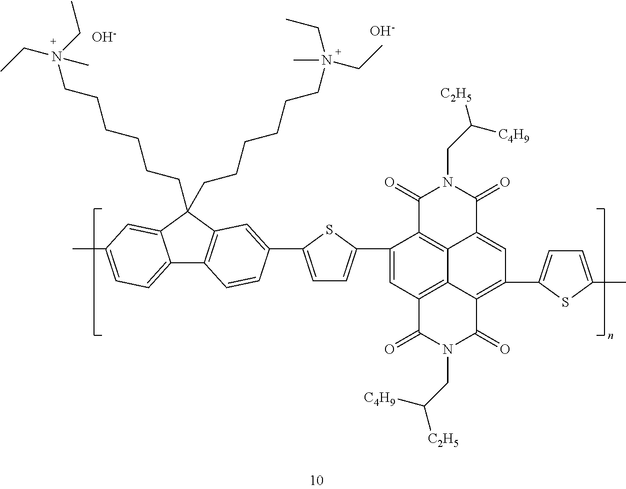

Putting 50 mg of PNDIT-F6N obtained in Example 1 in a 50 mL single-necked flask, adding 10 mL of clean chloroform and stirring it to get dissolved, then adding 0.5 mL of methyl iodide to the reaction solution, sealing, keeping in the dark, reacting at room temperature for 48 h, and adding DMSO to the reaction solution to dissolve a solid if the solid precipitates during the reaction. After completion of the reaction, concentrating, precipitating the solid in ethyl acetate, re-dissolving the solid, re-precipitating, filtering, and drying to obtain the product poly{2,7-[9,9′-bis(N,N-diethylhexyl-6-iodoamine)fluorene]-co-5,5%[2,6-(bis-2-thienyl)-N,N′-diisooctyl-1,4,5,8-naphthalimide]} (PNDIT-F6N-I).

First soaking the anion exchange resin IRN-78 sold by Acros Corporation in deionized water for 2 h, then filling it into a packed column with a sand board, dissolving 30 mg of the polymer PNDIT-F6N-I in 10 mL of DMSO, adding the mixture to an ion exchange resin column, passing the column with the mixed solvent of DMSO water (at a volume ratio of 1:1), concentrating the solution obtained from the column, precipitating in ethyl acetate, and drying to obtain the product poly{2,7-[9,9′-bis(N,N-diethylhexyl-6-hydroxylamine)fluorene]-co-5,5′-[2,6-(bis-2-thienyl)-N,N′-diisooctyl-1,4,5,8-naphthalimide]} (PNDIT-F6N-OH).

### Example 4

### Preparation of poly{2,7-[9,9′-bis(N,N′-dimethylpropyl-3-hydroxylamine)fluorene]-co-5,5′-[2,6-(bis-2-thienyl)-N,N′-diisooctyl-1,4,5,8-naphthalimide]} (PNDIT-F3N-OH)

The chemical reaction process is shown below, including the following specific reaction steps and reaction conditions:

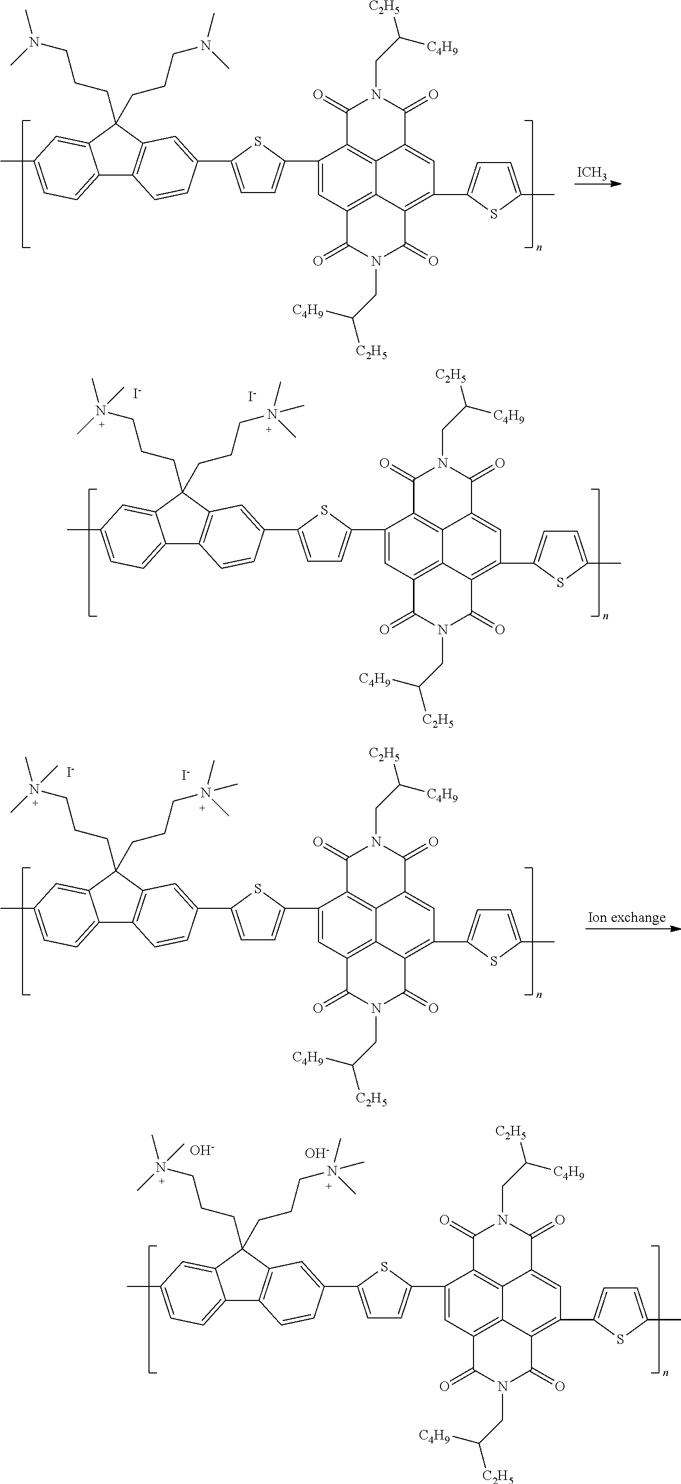

The specific implementation is the same as in Example 3, except that the raw material was PNDIT-F3N prepared in Example 2 instead of PNDIT-F6N prepared in Example 1.

### Example 5

### Preparation of poly{2,5-thiophen-co-N,N′-(4-[6-(diethylamino)hexyloxy]benzyl)-1,4,5,8-naphthalimide}} (PNDIPNT)

The chemical reaction process is shown below, including the following specific reaction steps and reaction conditions:

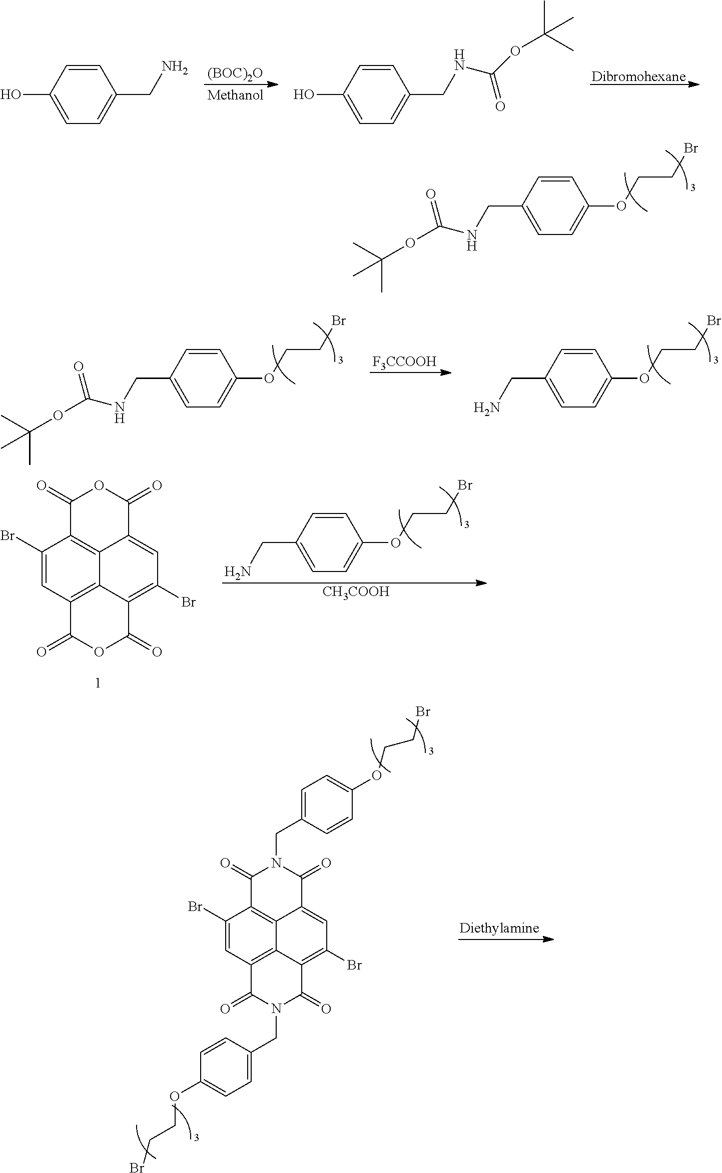

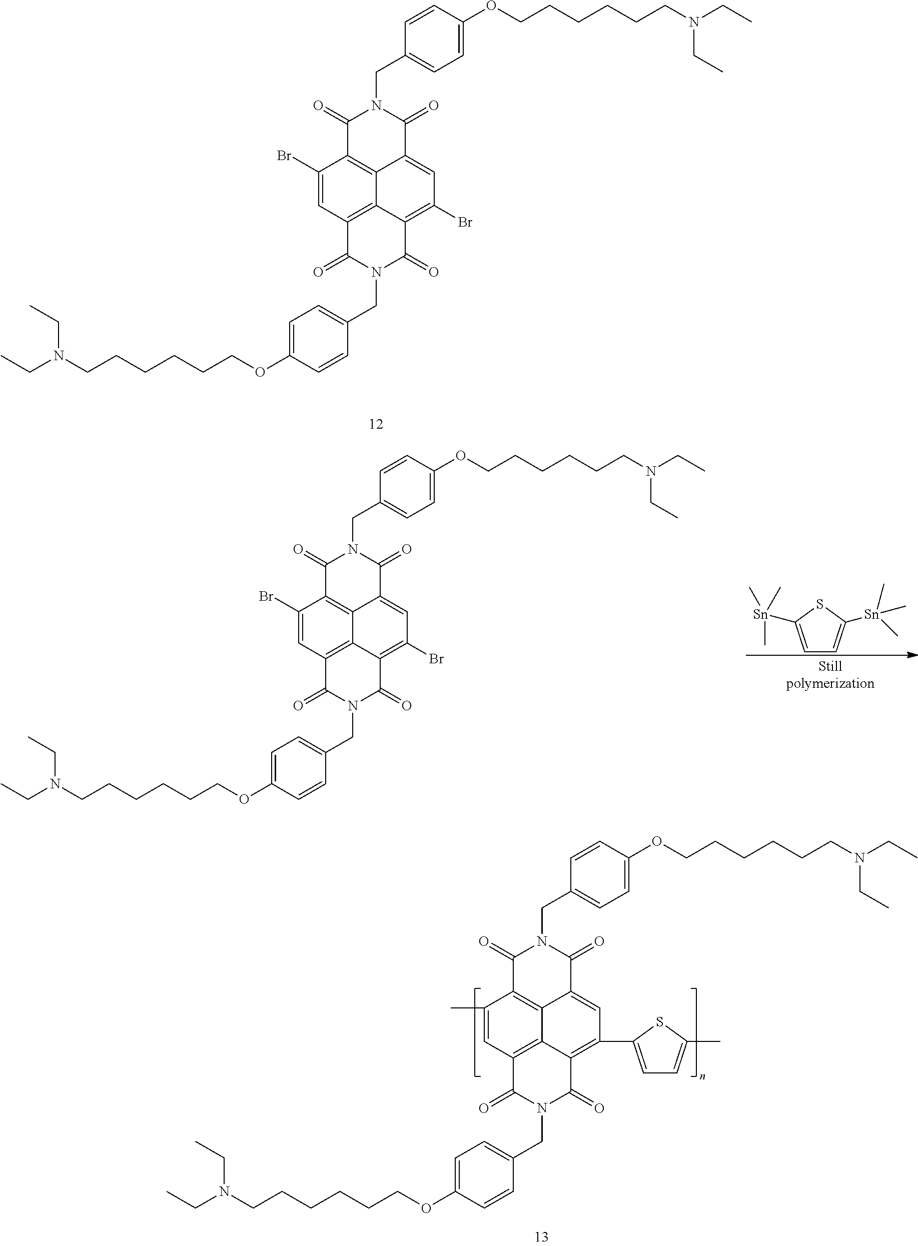

### (1) Preparation of the monomer N-tert-butyl-4-hydroxybenzylamine

Putting 12.3 g (100 mmol) of the monomer p-hydroxybenzylamine in a 100 mL two-necked flask equipped with a stir bar and bubbling nitrogen for 10 min, adding 40 mL of clean methanol to the reaction flask and stirring it to get dissolved, cooling the reaction flask in an ice bath to a solution temperature of 4° C. under the protection of nitrogen, slowly adding 26.2 g (120 mmol) of di-tert-butyl dicarbonate dropwise to the reaction solution, continuing the reaction for 2 h in the ice bath, concentrating after completion of the reaction, and distillating under reduced pressure to obtain 18.3 g of the product at a yield of 83%.

### (2) Preparation of the monomer 4-(6-bromohexyloxy)benzylamine

Adding 13.4 g (90 mmol) of the product obtained in the reaction (1) to a 500 mL three-necked flask equipped with a magnetic stir bar, bubbling nitrogen for 10 min, adding 100 mL of dibromohexane, adding 50 mL of a 2 mol/L KOH aqueous solution, adding 1 g of tetrabutylammonium bromide, and heating to reflux reaction for 6 h. After completion of the reaction, pouring the mixture into water, extracting and separating to obtain an organic phase, removing excess dibromohexane by distillating under reduced pressure to obtain the crude product, adding excess trifluoroacetic acid without purification directly to the dichloromethane solution containing the crude product and reacting for 3 h at room temperature, concentrating to obtain an oily liquid, dissolving the liquid in the methanol solution, neutralizing the solution with a 4 mol/L sodium hydroxide solution to be alkaline, extracting with dichloromethane to obtain an organic phase, drying with anhydrous magnesium sulfate, filtering, concentrating to obtain the crude product, and distillating under reduced pressure to obtain 20.0 g of the product at a yield of 78%.

### (3) Preparation of the monomer 2,6-dibromo-N,N′-bis[4-(6-bromohexyloxy)benzyl]-1,4,5,8-naphthalimide

The specific method of preparation is described with reference to the method disclosed in [PCT WO2011/144537 A1].

### (4) Preparation of the monomer 2,6-dibromo-N,N′-bis[4-(6-diethylamino)hexyloxy]benzyl]-1,4,5,8-naphthalimide

Adding 4.0 g (5 mmol) of the product obtained in the reaction (3) to a 100 mL one-necked flask equipped with a magnetic stir bar, adding 30 mL of clean dichloromethane, stirring, adding excess diethylamine under the protection of nitrogen, and reacting in the dark at room temperature for 36 h. After completion of the reaction, directly concentrating, drying, and passing a silica gel column to obtain 1.4 g of a yellow solid at a yield of 30%.

### (5) Preparation of poly{2,5-thiophen-co-N,N′-(4-[6-(diethylamino)hexyloxy]benzyl)-1,4,5,8-naphthalimide}} (PNDIPNT)

Adding 236.4 mg (0.25 mmol) of the product obtained in step (4) and 102.4 mg (0.25 mmol) of 2,5-ditrimethylthiophene to a 25 mL two-necked round-bottomed flask equipped with a magnetic stirrer, pumping ventilation with nitrogen for 3 times, injecting 10 mL of chlorobenzene into the reaction flask and stirring, adding 4 mg of a catalyst Pd₂(dba)₃ in the presence of nitrogen, adding 8 mg of a ligand P-(toyl)₃, and heating to 95° C. to react for 48 h. After the completion of the reaction, precipitating the reaction solution into methanol to obtain a crude product, filtering, drying, then extracting and washing the polymer with acetone, n-hexane and dichloromethane successively for 24 h, and then extracting 201.8 mg of the target polymer with chloroform at a yield of 87%. Mn=34000, and PDI=1.6.

### Example 6

The polymer materials obtained in Examples 1 to 4 (PNDIT-F6N; PNDIT-F3N; PNDIT-F6N-OH; PNDIT-F3N-OH) are taken as examples to illustrate that such polymer materials can be used as an electron transport layer in an organic solar cell.

Steps were carried out as follows: Using several pieces of ITO conductive glass having the square resistance of about 20 ohm/square at the specification of 15 mm×15 mm per piece. Ultrasonically cleaning them with acetone, a micron-scale semiconductor special-purpose detergent, deionized water and isopropanol in turn for more than half an hour, and putting them in a constant-temperature oven on standby. Before use, treating the ITO glass sheets with oxygen plasma for 4 min. Using a PEDOT:PSS (polyethylene dioxythiophene) aqueous dispersion (available from Bayer Corporation, Clevios P VP AI 4083) as the hole transport layer, and spin-coating it onto ITO with a homogenizer (KW-4A) at a high speed, with the thickness preferably about 40 nm, wherein the thickness is determined by solution concentration and rotational speed, and was measured and monitored with a surface profiler (Alpha-Tencor-500 of Tritek Corporation). After formation of a film, heating it in air at 150° C. for 20 min, and transferring it into a glove box on standby.

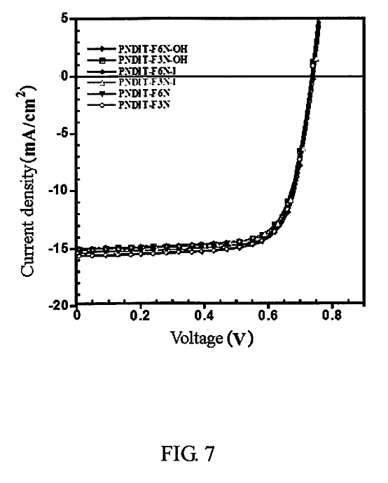

Weighing the active layer donor material conjugated polymer PTB7 and the acceptor material PC₇₁BM in a clean bottle (at a mass ratio of 1:1.5), transferring them into a nitrogen-protected film formation dedicated glove box (VAC Corporation), dissolving in a mixed solvent of chlorobenzene/1,8-diiodoctane (at a volume ratio of 100:3) at a concentration of 11 mg/mL, and throwing a 100 nm thick film over the PEDOT:PSS film. Putting the polymer materials obtained in the above Examples 1 to 4 in a clean vial, transferring into a nitrogen-protected film formation dedicated glove box, preparing a solution at a concentration of 0.5 mg/ml with the polar solvent methanol, and stirring well on a stirring station. Spin-coating the above solution on the active layer into an electron transport layer. Vacuum-depositing aluminum (80 nm) on the electron transport layer into an electron collection layer. All the preparations were carried out in a nitrogen-protected glove box. The current-voltage characteristics of the device were measured with a Keithley236 current-voltage measurement system and a calibrated silicon photodiode. The energy conversion efficiency of the device was measured with the standard solar spectrum AM1.5G simulator (Oriel model 91192). The energy of the simulated solar light was calibrated to 100 mW/cm² using a standard silicon cell before testing. The relationship between the current density and the voltage of the device under illumination is shown in FIG. 7, and the specific device efficiency is shown in Table 1.

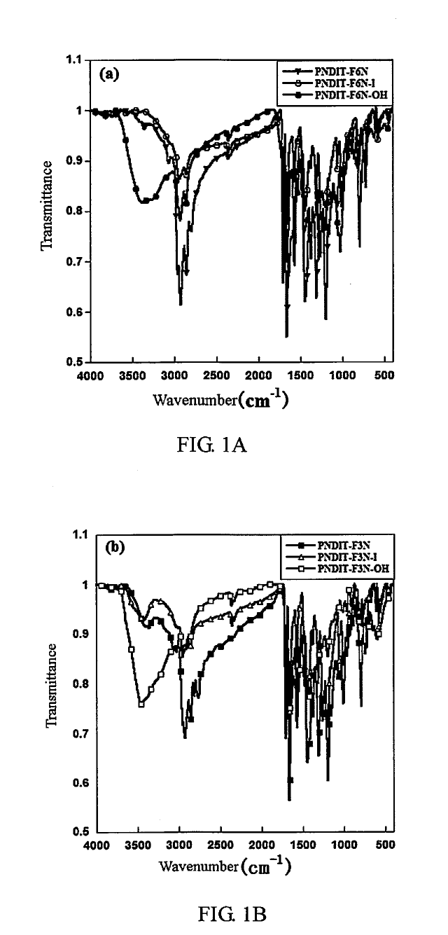

It can be seen from FIG. 1 that the infrared absorption spectra before and after quaternization are not significantly different; however, after ion exchange, with the iodine ions exchanged for the hydroxide ions, the infrared absorption peak of the hydroxyl radical is clearly observed on the IR spectrum.

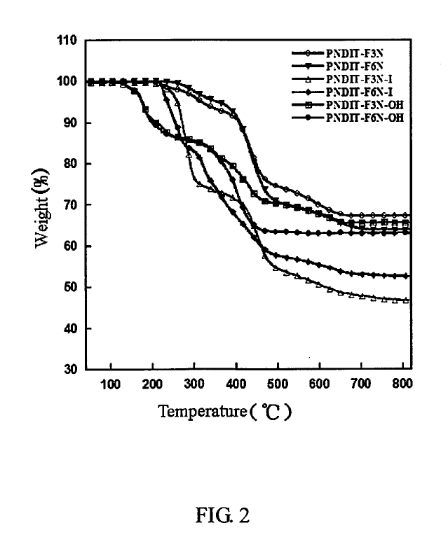

It can be seen from FIG. 2 that the decomposition temperatures of these polymer materials are 358° C., 216° C., 178° C., 332° C., 240° C. and 178° C., respectively.

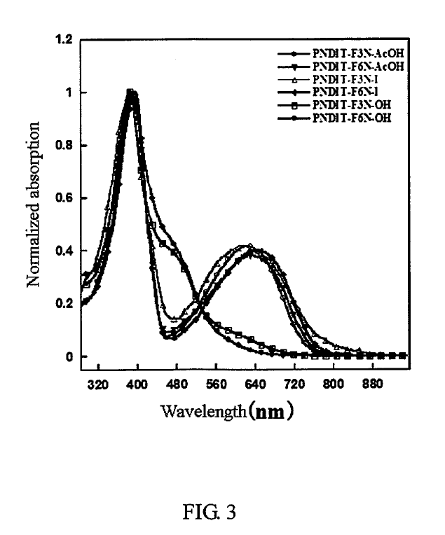

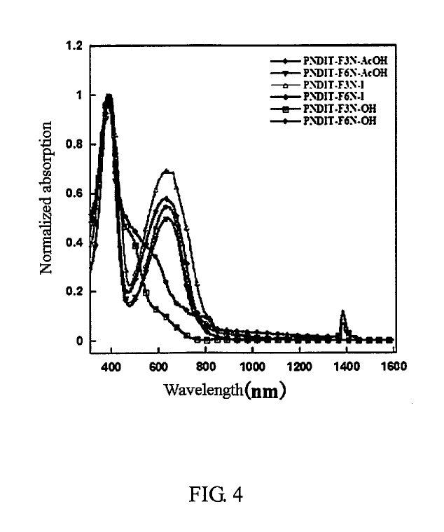

It is clear from FIGS. 3 and 4 that the absorption spectra of the hydroxyl-containing polymers are much different from the absorption spectra of the two precursor polymer materials to which they themselves correspond, showing the phenomenon that the conjugated main chain is n-type doped, indicating that the materials of the examples of the present invention can effect a change in the absorption spectrum of the polymer material by introducing a different pair of ions into the amine group.

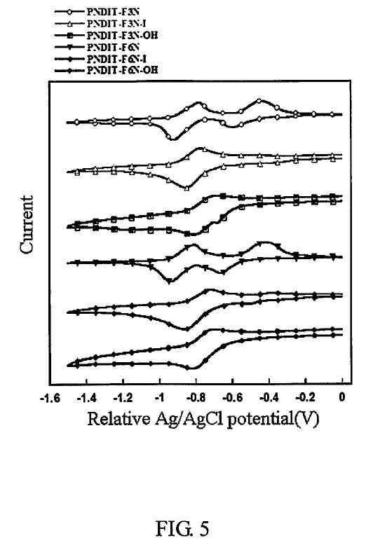

It can be seen from FIG. 5 that the LUMO levels of these polymer materials are very low, and these polymer materials have a relatively matching energy level with the electron acceptor material in the active layer, wherein the reduction potential of the hydroxide-containing polymers are lower than the reduction potential of the two precursor polymers to which they themselves correspond, which indicates that the ability of the hydroxide-containing polymer materials to be reduced is weaker than that of the two precursor polymers to which they themselves correspond, with the corresponding LUMO energy level also higher. The LUMO levels of these polymer materials are −3.81 eV, −3.91 eV, −3.83 eV, −3.90 eV, −3.97 eV and −3.83 eV, respectively.

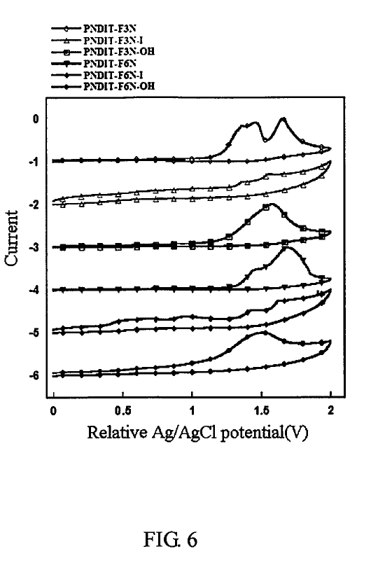

It can be seen from FIG. 6 that the polymer materials of the neutral amine with different alkyl chains connected to the fluorene are different in the oxidation potential, and the oxidation potential of the hydroxide-containing polymers are lower than the oxidation potential of the two precursor polymers to which they themselves correspond, which indicates that the ability of the hydroxide-containing polymer materials to be oxidized is stronger than that of the two precursor polymers to which they themselves correspond, with the corresponding HOMO energy level also higher.

FIG. 7 is a J-V diagram of a solar cell device prepared by using PTB7:PC₇₁BM as the active layer and the polymers obtained in Examples 1 to 4 as the electron transport layer, with the related device performance as shown in Table 1.

Table 1 shows the performance of the solar device using the PTB7:PC₇₁BM (at a mass ratio of 1:1.5) as the active layer and the polymers obtained in Examples 1 to 4 as the electron transport layer.

The structure of the device is: ITO/PEDOT:PSS/PTB7:PC₇₁BM/electron transport layer (5 nm)/A1

TABLE 1

| Electron | | | | |
|---|---|---|---|---|
| transport layer | V_{oc} (V) | J_{sc} (mA/cm²) | FF (%) | PCE (%) |
| PNDIT-F3N | 0.74 | 15.63 | 72.87 | 8.4 |
| PNDIT-F6N | 0.74 | 15.39 | 73.03 | 8.3 |
| PNDIT-F3N-I | 0.74 | 15.34 | 72.78 | 8.3 |
| PNDIT-F6N-I | 0.74 | 15.59 | 73.12 | 8.4 |
| PNDIT-F3N-OH | 0.73 | 15.05 | 73.65 | 8.1 |
| PNDIT-F6N-OH | 0.74 | 14.96 | 73.85 | 8.2 |

Wherein Voc is the open circuit voltage, Jsc is the short-circuit current, FF is the filling factor, and PCE is the power conversion efficiency.

It can be seen that the device using the n-type water-alcohol soluble conjugated polymer material containing the naphthalimide ring as the electron transport layer exhibits excellent device performance, indicating that these materials have excellent interface modification properties.

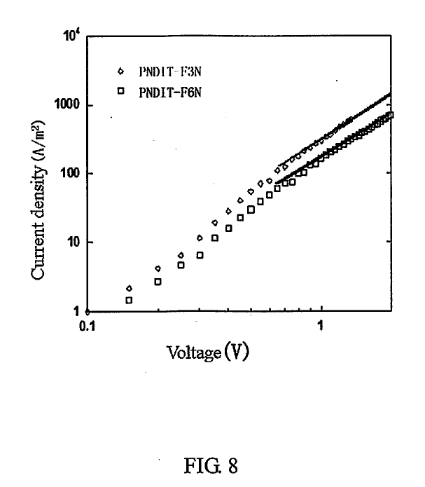

FIG. 8 is a characterization diagram of the single-electron device of the polymers PNDIT-F6N and PNDIT-F3N obtained in Examples 1 and 2, with the related device performance as shown in Table 2.

Table 2 shows the device performance of the single-electron device of the polymers obtained in Examples 1 and 2.

The structure of the device is ITO/Al/polymer (100 nm)/Al

TABLE 2

| Polymer | Electron mobility |
|---|---|
| PNDIT-F3N | 1.9 × 10⁻⁴ cm²V⁻¹s⁻¹ |
| PNDIT-F6N | 1.1 × 10⁻⁴ cm²V⁻¹s⁻¹ |

It can be seen that the n-type water-alcohol soluble conjugated polymer material containing the naphthalimide ring has high electron mobility, indicating that it has the potential to be an electron transport layer allowing thick film processing in an organic thin film battery device process.

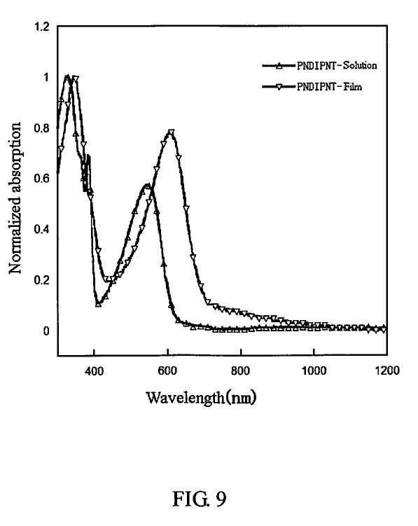

It can be seen from FIG. 9 that the thin film absorption spectrum of the polymer PNDIPNT has obviously more red shift than that of the solution, wherein the charge transfer absorption peak in the molecule red-shifts more obviously from the original 542 nm to 607 nm, indicating that the polymer is well accumulated in the thin film state, which is conducive to the transmission of electrons.

### Example 7

Preparation of the Organic-Inorganic Planar Heterojunction Perovskite Solar Cells:

Using several pieces of ITO conductive glass having the square resistance of about 20 ohm/square at the specification of 15 mm×15 mm per piece. Ultrasonically cleaning them with acetone, a micron-scale semiconductor special-purpose detergent, deionized water and isopropanol in turn for more than half an hour, and putting them in a constant-temperature oven on standby. Before use, treating the ITO glass sheets with oxygen plasma for 4 min. Using a PEDOT:PSS (polyethylene dioxythiophene) aqueous dispersion (available from Bayer Corporation, Clevios P VP AI 4083) as the hole transport layer, and spin-coating it onto ITO with a homogenizer (KW-4A) at a high speed, with the thickness preferably about 40 nm, wherein the thickness is determined by solution concentration and rotational speed, and was measured and monitored with a surface profiler (Alpha-Tencor-500 of Tritek Corporation). After formation of a film, heating it in air at 150° C. for 20 min, and transferring it to a glove box on standby.

Blending and dissolving CH₃NH₃I, PbI₂ and PbCl₂ (at a ratio of 4:1:1) in 1 mL of DMF to prepare a solution at a mass fraction of 40%, heating up to 60° C., stirring for 12 h to obtain a photoactive layer material precursor solution, spin-coating the solution onto the PEDOT:PSS layer at a rotational speed of 3000 rpm, and then annealing at 100° C. for 1 h. The electron transport layer was obtained by spin-coating a layer of PNDIT-F6N solution onto the surface of the photoactive layer; dissolving PNDIT-F6N in Example 1 in a solvent such as chlorobenzene, dichlorobenzene, toluene, chloroform, and xylene to prepare an electron transport layer solution in the concentration range from 1 to 60 mg/ml. The film thickness of PNDIT-F6N is usually 50 to 200 nm. At the same time, PC_{6I}BM (dissolved in chlorobenzene, 30 mg/ml) was used as the electron transport material in the contrast device. Finally, a layer of a silver electrode was deposited by vapor deposition.

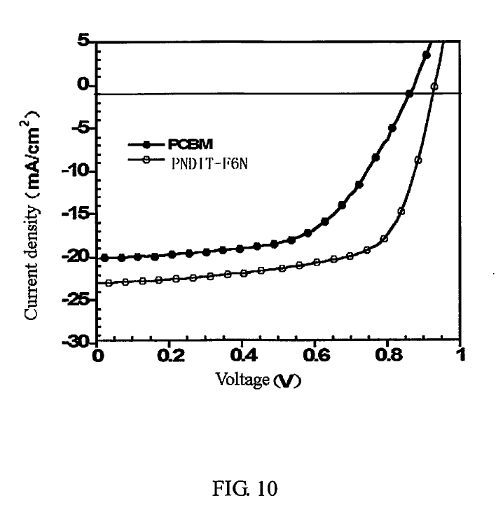

It can be seen from FIG. 10 that the short-circuit current (J_{sc}) and the open circuit voltage (V_{oc}) of the best device having the structure of ITO/PEDOT/Perovskite/PNDIT-F6N/Ag are 22.8 mA/cm² and 0.93 V, respectively. While PC6IBM was used as the electron transport layer in the contrast device, with V_{oc} and the filling factor (FF) decreased significantly.

The efficiency of the specific solar cell device is shown in Table 3.

It can be seen from Table 3 that, with the new material PNDIT-F6N substituting for the commonly used electron transport layer PC₆₁BM, V_{oc}, J_{sc} and FF are significantly improved, and the device efficiency is increased from 10.1% to about 14%, which indicates a more balanced transport of carriers across the device. In particular, since the PNDIT-F6N material contains strongly polar groups, it can reduce the work content of a metal when in contact with the metal, thus significantly modifying the interface and increasing the open circuit voltage.

TABLE 3
Structure of the device: ITO/PEDOT/Perovskite/ETL/Ag

| Interface layer of | | | | |
|---|---|---|---|---|
| the cathode | V_{oc} (V) | J_{sc} (mA/cm²) | FF (%) | PCE (%) |
| PC₆₁BM | 0.88 | 20.1 | 57.1 | 10.1 |
| PNDIT-F6N | 0.93 | 22.7 | 64.0 | 14.0 |

It can be seen that the introduction of the electron transport layer material of the present invention into a perovskite solar cell can significantly improve the carrier transport properties of the perovskite solar cells, increase the open circuit voltage of the device, and remarkably improve the photoelectric conversion performance of the device.

The above examples are preferred embodiments of the present invention; however, the embodiments of the present invention are free from restriction of the above examples, and any other alteration, modification, substitution, combination and simplification without departing from the spiritual essence and principle of the present invention are equivalent replacements and fall within the scope of protection of the present invention.

What is claimed is:

1. A material suitable as an electron transport material, characterized by the following structure: 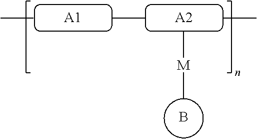
wherein n is a natural number of 1 to 10,000, B is a strongly polar group, A1 and A2 are the same or different aromatic ring derivatives or conjugated units containing carbon-carbon double bonds and carbon-nitrogen bonds, M is a connection unit between A2 and B and is an alkyl group containing 1 to 20 carbon atoms, or is an alkyl group in which one or more carbon atoms are replaced by one or more functional groups selected from oxygen atoms, alkenyl groups, alkynyl groups, aryl groups or ester groups, and the hydrogen atom is replaced by a fluorine atom, a chlorine atom, a bromine atom, an iodine atom or the above-mentioned functional groups.

2. The material according to claim 1, wherein the strongly polar group is selected from one or more of the group consisting of an amine group, a quaternary ammonium salt group, a phosphate radical, a phosphate group, a sulfonate radical, a carboxyl group and a hydroxyl group.

3. The material according to claim 1, wherein A1 and A2 comprise one or more of the following structures: 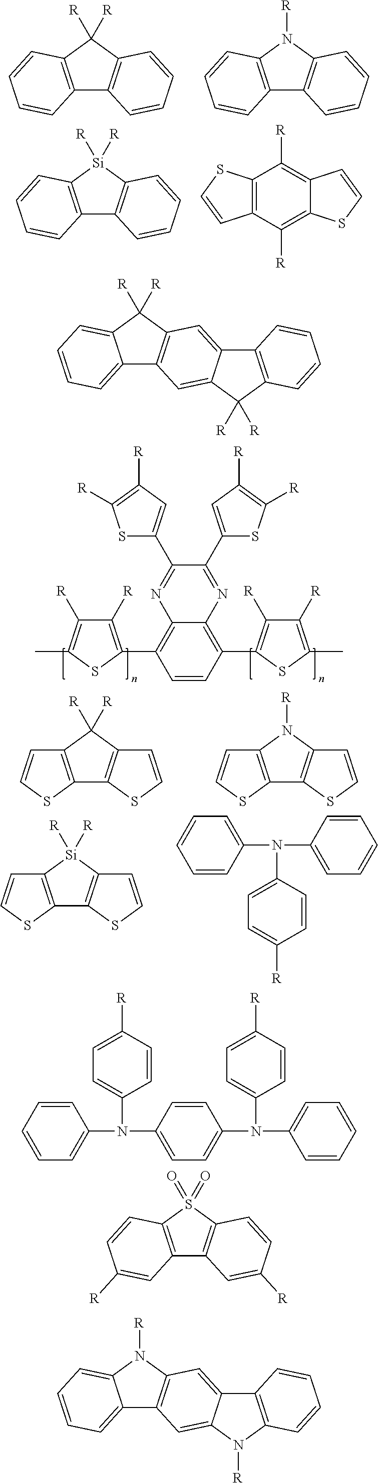 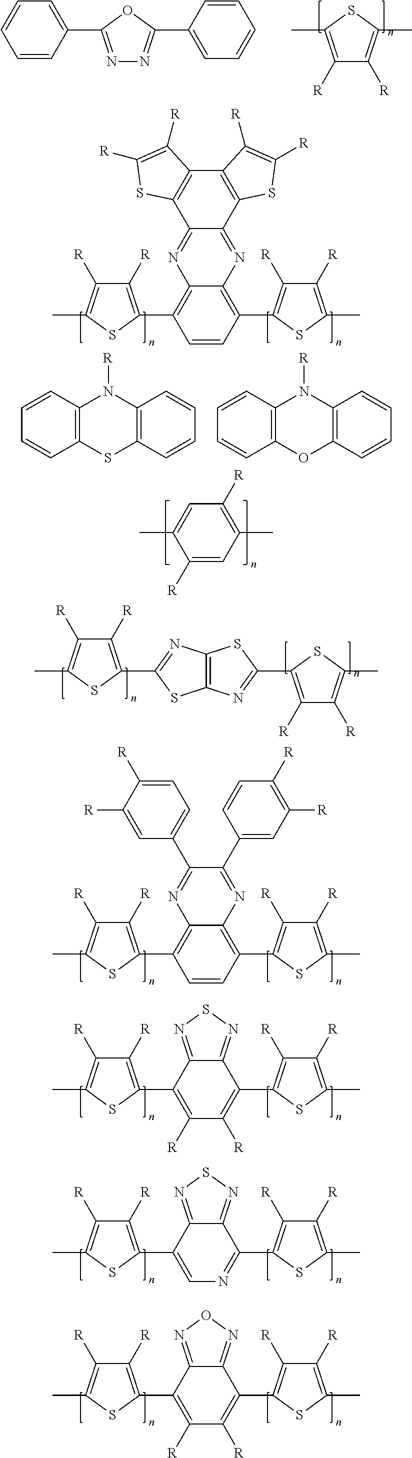 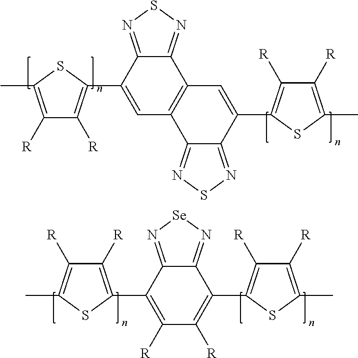 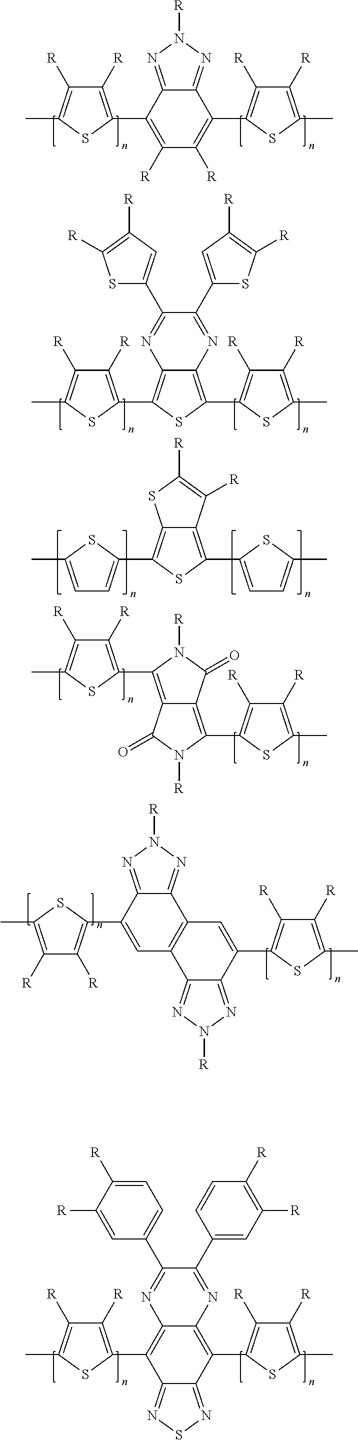 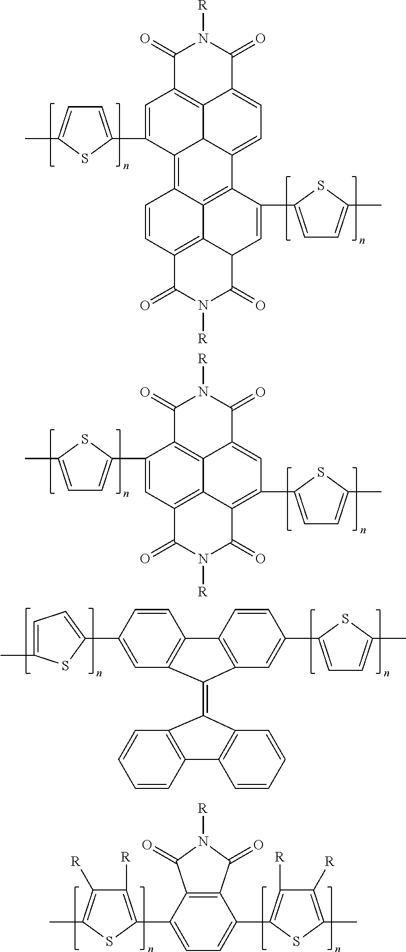
wherein n is a natural number of 1 to 10000, R is an alkyl group containing 1 to 20 carbon atoms, or is an alkyl group in which one or more carbon atoms are replaced by one or more functional groups selected from oxygen atoms, alkenyl groups, alkynyl groups, aryl groups or ester groups, and the hydrogen atom is replaced by a fluorine atom, a chlorine atom, a bromine atom, an iodine atom or the above-mentioned functional groups.

4. The material according to claim 1, wherein
A1 is of the structure 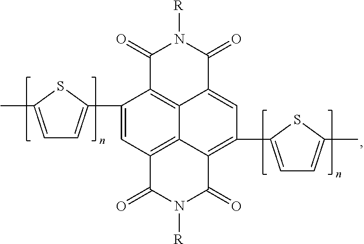
wherein n is a natural number of 1 to 3, R is an alkyl group containing 1 to 20 carbon atoms, or is an alkyl group in which one or more carbon atoms are replaced by one or more functional groups selected from oxygen atoms, alkenyl groups, alkynyl groups, aryl groups or ester groups, and the hydrogen atom is replaced by a fluorine atom, a chlorine atom, a bromine atom, an iodine atom or the above-mentioned functional groups;
A2 comprises one or more of the following structures: 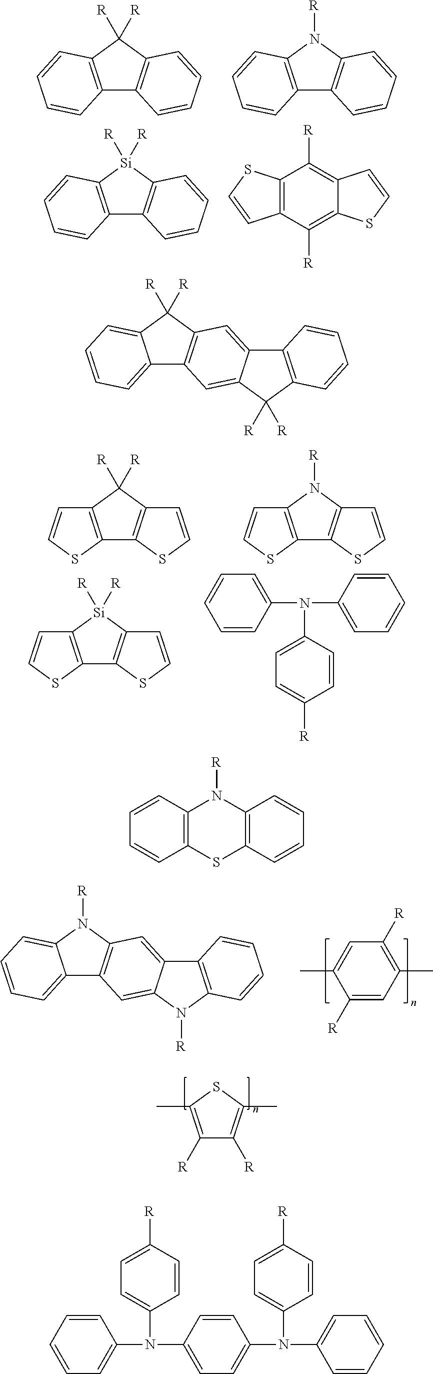
wherein n is a natural number of 1 to 10000, R is an alkyl group containing 1 to 20 carbon atoms, or is an alkyl group in which one or more carbon atoms are replaced by one or more functional groups selected from oxygen atoms, alkenyl groups, alkynyl groups, aryl groups or ester groups, and the hydrogen atom is replaced by a fluorine atom, a chlorine atom, a bromine atom, an iodine atom or the above-mentioned functional groups.

5. A method for preparing a material suitable as an electron transport material comprising: mixing A2 monomers containing a polar group and monomers of a naphthalimide ring in an equimolar amount to obtain a polymer in which the polar groups are not ionized under the action of an organic base and a palladium catalyst;
subjecting the resulting polymer to a salinization reaction at room temperature in the dark to obtain a polymer in which the polar groups are ionized;
treating the resulting ionized polymer with an ion exchange resin containing different pairs of ions; and
subjecting the ionized polymer treated with the ion exchange resin to a separation to obtain a polymer material wherein the polymer material is an electron transporting material.

6. An optoelectronic device containing an electron transport layer comprised of the material as described by claim 1.

7. The optoelectronic device of claim 6, wherein the device is an organic solar cell or a perovskite solar cell.

8. The optoelectronic device of claim 6, wherein the device is a single electron device.

9. The optoelectronic device of claim 6, wherein the material further comprises a polymer, the polymer selected from the group consisting of: poly{2,7-[9,9′-bis(N,N-diethylhexyl-6-amino)fluorene]-co-5,5′-[2,6-(bis-2-thienyl)-N,N′-diisooctyl-1,4,5,8-naphthalimide]}; poly{2,7-[9,9′-bis(N,N-dimethylpropyl-3-amino)fluorene]-co-5,5′-[2,6-(bis-2-thienyl)-N,N′-diisooctyl-1,4,5,8-naphthalimide]}; poly{2,7-[9,9′-bis(N,N-diethylhexyl-6-hydroxylamine)fluorene]-co-5,5′-[2,6-(bis-2-thienyl)-N,N′-diisooctyl-1,4,5,8-naphthalimide]}; and poly{2,7-[9,9′-bis(N,N-dimethylpropyl-3-hydroxylamine)fluorene]-co-5,5′-[2,6-(bis-2-thienyl)-N,N′-diisooctyl-1,4,5,8-naphthalimide]}
